SEQUENCE LISTING (14 sequences)

SEQ ID NO: 1 - DNA, 152 bp, Artificial Sequence
  a nucleotide sequence against murine Actb gene
caacgaaggagctgcaaagaagctgtgctcgcgggtggacgcgactctcgttggcccccgaccgttacagactgttctcagttcgttggcccccgaccgttacagactgttctcagttcgttggcccccgaccgttacagactgttctcagt

SEQ ID NO: 2 - DNA, 155 bp, Artificial Sequence
  a second nucleotide sequence against murine Actb gene
ggtggcttttgggagggtgagggacttcctgtaaccacttatttcatggatcgttggcccccgaccgttacagactgttctcagttcgttggcccccgaccgttacagactgttctcagttcgttggcccccgaccgttacagactgttctcagt

SEQ ID NO: 3 - DNA, 155 bp, Artificial Sequence
  a nucleotide sequence against murine Actg gene
ctccccagcccccaagtgaccgagccacatgaactaaggactaaatcaagtctataaacgagcaattacataagacatccgtagatctataaacgagcaattacataagacatccgtagatctataaacgagcaattacataagacatccgtaga

SEQ ID NO: 4 - DNA, 150 bp, Artificial Sequence
  a second nucleotide sequence against murine Actg gene
tgacgagtgcggcgatttcttcttccattgcgatcggcgaaggactctataaacgagcaattacataagacatccgtagatctataaacgagcaattacataagacatccgtagatctataaacgagcaattacataagacatccgtaga

SEQ ID NO: 5 - DNA, 160 bp, Artificial Sequence
  a nucleotide sequence for a secondary nucleic acid element for hybridization with Actb primary nucleic acid element
actgagaacagtctgtaacggtcgggggccaacgaacgcgattgactaccagactatacgacgcgattgactaccagactatacgacgcgattgactaccagactatacgacgcgattgactaccagactatacgacgcgattgactaccagactatacg

SEQ ID NO: 6 - DNA, 160 bp, Artificial Sequence
  a nucleotide sequence for a secondary nucleic acid element for hybridization with Actg primary nucleic acid element
tctacggatgtcttatgtaattgctcgtttatagataccaattctgacatatgtgactcataccaattctgacatatgtgactcataccaattctgacatatgtgactcataccaattctgacatatgtgactcataccaattctgacatatgtgactca

SEQ ID NO: 7 - DNA, 25 bp, Artificial Sequence
  a nucleotide sequence for a tertiary nucleic acid element for hybridization with Actb secondary nucleic acid element
cgtatagtctggtagtcaatcgcgt

SEQ ID NO: 8 - DNA, 25 bp, Artificial Sequence
  a nucleotide sequence for a tertiary nucleic acid element for hybridization with Actg secondary nucleic acid element
tgagtcacatatgtcagaattggta

SEQ ID NO: 9 - DNA, 155 bp, Artificial Sequence
  a nucleotide sequence for recognition of the Nrg1-III gene
tatgttccgctgccggaagcccatcgagagatgggtctgcactcagctgatcgttggcccccgaccgttacagactgttctcagttcgttggcccccgaccgttacagactgttctcagttcgttggcccccgaccgttacagactgttctcagt

SEQ ID NO: 10 - DNA, 153 bp, Artificial Sequence
  a second nucleotide sequence for recognition of the Nrg1-III gene
agatcttctcggagttgaggcaccctctgagacgctccgcttccaggctcgttggcccccgaccgttacagactgttctcagttcgttggcccccgaccgttacagactgttctcagttcgttggcccccgaccgttacagactgttctcagt

SEQ ID NO: 11 - DNA, 155 bp, Artificial Sequence
  a third nucleotide sequence for recognition of the Nrg1-III gene
cccccagggtcaaggtgggtaggagagtcgtattcgaatatcttgtccactcgttggcccccgaccgttacagactgttctcagttcgttggcccccgaccgttacagactgttctcagttcgttggcccccgaccgttacagactgttctcagt

SEQ ID NO: 12 - DNA, 155 bp, Artificial Sequence
  a nucleotide sequence for recognition of the murine ChAT gene
ctcgctcccaccgcttctgcaaactccacagatgaggtctctttgcagcctctataaacgagcaattacataagacatccgtagatctataaacgagcaattacataagacatccgtagatctataaacgagcaattacataagacatccgtaga

SEQ ID NO: 13 - DNA, 155 bp, Artificial Sequence
  a second nucleotide sequence for recognition of the murine ChAT gene
aacatgccagcttcatgtgagcccccaaggataggggagcagcaacaagctctataaacgagcaattacataagacatccgtagatctataaacgagcaattacataagacatccgtagatctataaacgagcaattacataagacatccgtaga

SEQ ID NO: 14 - DNA, 153 bp, Artificial Sequence
  a third nucleotide sequence for recognition of the murine ChAT gene
gggggttataacaggctccatacccattgggtaccacagggccataactctataaacgagcaattacataagacatccgtagatctataaacgagcaattacataagacatccgtagatctataaacgagcaattacataagacatccgtaga

(19) United States
(12) Patent Application Publication
(10) Pub. No.: US 2015/0105298 A1
(43) Pub. Date: Apr. 16, 2015

(54) MULTI-OLIGOMER IN SITU HYBRIDIZATION PROBES

(71) Applicant: The Research Foundation for The State University of New York, Albany, NY (US)

(72) Inventor: Kevin Czaplinski, Huntington, NY (US)

(73) Assignee: The Research Foundation for The State University of New York, Albany, NY (US)

(21) Appl. No.: 14/511,315

(22) Filed: Oct. 10, 2014

Related U.S. Application Data
(60) Provisional application No. 61/889,080, filed on Oct. 10, 2013.

(51) Int. Cl. C12Q 1/68

(52) U.S. Cl. CPC C12Q 1/6876
USPC 506/16; 506/23; 536/24.3

(57) ABSTRACT

The disclosure relates to in situ hybridization probes for the detection of target nucleic acid sequences within a sample and methods of making and using the same. The in situ hybridization probes of the current disclosure include a plurality of nucleic acid elements capable of selectively hybridizing to at least a portion of a nucleic acid of interest and/or other nucleic acid elements of the in situ hybridization probe, which enable the detection of a target nucleic acid. The current disclosure also relates to kits which incorporate the in situ hybridization probe compositions of the instant disclosure.

### CROSS REFERENCE TO RELATED APPLICATIONS

This application claims priority from U.S. Provisional Application No. 61/889,080 filed Oct. 10, 2013, the entire contents of which are incorporated herein by reference.

### FIELD OF THE DISCLOSURE

The present disclosure relates to compositions, methods and kits concerning the identification, and detection of nucleic acids. The present disclosure includes compositions that can be incorporated into kits for the detection of nucleic acids. The present disclosure also includes methods for detecting the presence of nucleic acids in a sample.

### INCORPORATION BY REFERENCE OF SEQUENCE LISTING

The Sequence Listing in the ASCII text file, named as 29959_SequenceListing.txt of 6 kilobytes, created on Oct. 6, 2014, and submitted to the United States Patent and Trademark Office via EFS-Web, is incorporated herein by reference.

### BACKGROUND OF THE DISCLOSURE

In vitro transcribed in situ hybridization (ISH) probes are well known in the art and have noted applications in histology, and whole mount gene expression pattern analysis, but known limitations when applied to the detection of single nucleic acid molecules. See Qian, X. and Lloyd, R. V. (2003) Diagn. Mol. Pathol., 12(1):1-13; and Itzkovitz, S. and van Oudenaarden, A. (2011) Nat. Methods, 8(4 Suppl): S12-9. For example, it is commonly known that the lower limit for detecting an RNA species is about 20 copies per cell. However, in practice the limit of detection, due to the sensitivity of currently available assays, is generally found to be around 50 to 60 copies per cell. This limitation hinders the field of research significantly because approximately 80% of mRNAs are present at fewer than 10 copies per cell and approximately 95% of mRNAs are present in cells at fewer than 50 copies.

The synthesis of fluorescence in situ hybridization (FISH) probes comprised of fluorescently labeled nucleic acid molecules currently requires in house DNA synthesis and post-synthesis dye coupling, which is an inefficient process that is difficult to control. Limitations to synthesis of commercial probes commercially manifest from the increased probability of premature truncation as oligodeoxynucleotides (ODN) size increases during synthesis, which goes from 3′ to 5′. As a practical matter, the expense and difficulty involved in probe design makes the development of properly labeled nucleic acid molecules for use as FISH probes inaccessible to most laboratories. Additionally, quantitative gene probes with sequential nucleic acid probes that use branched nucleic acid probes to amplify signals are used to detect single nucleic acid molecules, but are very costly and time consuming to produce. See Player, A. N., et al., (2001) J. Histochem. Cytochem. 49(5): 603-12; and Collins, M. L., et al., (1997) Nucleic Acids Res. 25(15): 2979-84. Thus, alternative methods of making FISH more accessible and sensitive are highly sought-after.

The in situ hybridization probes described in the current disclosure, namely Fluorescence In Situ Hybridization with Sequential Tethered and Intertwined nucleic acid molecule Complexes (FISH-STICs) overcome the above noted limitations and permit rapid, simple, cost efficient and sensitive detection of multiple nucleic acids (and/or other nucleic acids) simultaneously. The methods and compositions of the current disclosure increases the fluorescence output of small stretches of RNA that can be recognized by multiply labeled nucleic acid molecules, making small stretches of nucleic acid more easily detectable.

### SUMMARY OF THE DISCLOSURE

Compositions for detecting a nucleic acid of interest are presented, including associated methods of making and using the same, as well as kits and systems incorporating the in situ hybridization probe compositions of the current disclosure.

The in situ hybridization probes of the current disclosure include one or more primary (first), a secondary (second) and a tertiary (third) nucleic acid each consisting of between 5 and 200 nucleotides, 10 and 175 nucleotides, or 20 and 160 nucleotides, 100 and 160 nucleotides, 25 and 50 nucleotides, 25 and 35 nucleotides, and 150 and 160 nucleotides, inclusive. These nucleic acid elements are sequentially hybridized to a sample containing nucleic acid and detected by methods currently known to the skilled artisan, detection of a probe or probes present on the tertiary nucleic acid element.

Another embodiment of the current disclosure is a method to provide in situ hybridization probes for the detection of target nucleic acid sequences. Generally, placement of an antisense hybridizing sequence at the 5′ end of a probe oligonucleotide molecule prevents any truncated oligodeoxynucleotides from hybridizing, and thus such truncated nucleic acid elements are unable to interfere with the successful hybridization of full-length probes. In one embodiment of the present disclosure, three sequential oligos are used as FISH-STIC probes in order to overcome the deficiencies disclosed in the art pertaining to the use of quintuply labeled single 50-mer nucleic acid elements (i.e. primary nucleic acid element). Next, 25-35 nucleotide intermediate (i.e., secondary) nucleic acid element sequences were developed because these oligos are large enough to accommodate decreasing hybridization stringency during successive steps. However, in certain embodiments intermediate tag sequences that vary in length have also been created and used in the current methods. Notably, the nucleic acid element sequences generated using the present methods have been developed through a random sequence generator and screened (e.g. BLAST search) to limit background complementarity, so that the methods disclosed herein can be adapted to use any sequence that is not highly complementary to an existing endogenous nucleotide sequence.

In certain embodiments, the disclosed methods include; (1) selecting at least one nucleic acid of interest present in a sample; (2) designing a primary nucleic acid including a nucleotide sequence located at the 5′end of the primary nucleic acid that is complimentary to a portion of the nucleic acid of interest and capable of hybridizing to such nucleic acid of interest, wherein said primary nucleic acid includes at least one recognition element; (3) designing a secondary nucleic acid including at least one annealing element located at the 5′ end of the second nucleic acid that is complimentary to the nucleotide sequence of the of recognition elements present in the primary nucleic acid, and a plurality of detection elements; (4) designing a tertiary nucleic acid including a nucleotide sequence complementary to the nucleic acid sequence of the detection element(s) present in the secondary nucleic acid, and a detection probe; (5) the primary, secondary and tertiary nucleic acid elements are then sequentially hybridized to the nucleic acid of interest. The sequential hybridization step can be carried out under conditions that prevent dissociation of previously hybridized nucleic acid element, which are well known in the art.

The compositions and methods of the current disclosure to employ oligonucleotides as nucleic acid-FISH probes for detection of nucleic acids (e.g., RNA or DNA) in cultured cells. The current compositions and methods facilitate the binding of a high concentration of detection probes to a short stretch of nucleotides using, for example, commercial DNA synthesis outlets available to any lab.

### BRIEF DESCRIPTION OF DRAWINGS AND TABLES

The file of this patent contains at least one drawing executed in color. Copies of this patent with color drawing(s) will be provided by the Patent and Trademark Office upon request and payment of the necessary fee.

FIGS. 1A-B. Sequential Hybridization Diagram. Primary Nucleic Acid Element Design and Synthesis. FIG. 1A) First, 50 nucleotides at the 5′ end of a primary nucleic acid element is designed and synthesized to be complementary to the target RNA (e.g., mRNA, ncRNA, miRNA). Then three repeats of a unique 35 nucleotide sequence (recognition elements) are added at the 3′ end of the primary nucleic acid element immediately following such first 50 nucleotides. The primary nucleic acid element is then hybridized to the target nucleic acid present in a sample. Secondary Nucleic Acid Design and Synthesis. The secondary nucleic acid element is comprised of at least 35 nucleotides at the 5′ end of the secondary nucleic acid (annealing elements) that are complementary to the 35 nucleotide sequence(s) of the primary nucleic acid (recognition elements). Additionally, the secondary nucleic acid element includes about five repeats of a distinct unique 20 to 25 nucleotide sequence added at the 3′ end of the secondary nucleic acid element (detection elements). A plurality of secondary nucleic acid elements are then hybridized to the sample previously hybridized to the primary nucleic acid elements, so that the annealing elements present in the secondary nucleic acid element will anneal or hybridize to the recognition elements of primary nucleic acid element. Tertiary Nucleic Acid Element Design and Synthesis. The tertiary nucleic acid element is designed to be complementary to the 20 to 25 nucleotide sequence(s) of the secondary nucleic acid element (detection elements) and synthesized with a probe (e.g., fluorescent dye, or any other means of detection) coupled to the 5′ end. The tertiary nucleic acid element is then applied to the sample and hybridized to the detection elements previously hybridized to the sample. Through these three sequential hybridizations the individual nucleic acid complexes, which include many tertiary nucleic acid elements, provide a bright signal for epifluorescence imaging and target nucleic acid detection. Multiple primary nucleic acid elements against the same mRNA can incorporate common secondary and tertiary nucleic acid elements, to increase brightness of the probes. Nucleic acid elements (ODN) shown are not drawn to scale. FIG. 1B) Probe design and hybridization methods. Three successive nucleic acid elements are sequentially hybridized to build a fluorescent probe on a targeted endogenous oligonucleotide sequence in fixed cells in situ. The primary nucleic acid element for a first hybridization has 50 nucleotides that are anti-parallel to the target endogenous molecule, and has three copies of a 35 nucleotide recognition element. Multiple primary nucleic acid elements that target different sequences within the same target molecule (e.g., DNA or RNA) can contain the same 35 nucleotide recognition elements to make target detection more robust. The secondary nucleic acid element for a second hybridization has a 35 nucleotide portion that is anti-parallel to the 35 nucleotide recognition element in the primary nucleic acid element, hybridizing in three copies per primary nucleic acid elements, as well as five copies of a 25 nucleotide detection element. The tertiary nucleic acid elements for a third hybridization are synthesized to contain a portion that is anti-parallel to the 25 nucleotide detection elements of the secondary nucleic acid element and has a fluorophore, which is covalently attached to the 5′ end of the tertiary nucleic acid element. In certain embodiments hybridization can occur in 5 copies per secondary nucleic acid element and up to and including 15 copies per individual primary nucleic acid element.

FIG. 2. Detection of Actb mRNA with a single in situ hybridization probes. An Actb primary nucleic acid element was hybridized to primary MEF cells. Top row: Normalized Cy3 images of cells after hybridization with complete FISH probes (A) or in situ hybridization probes lacking one element as indicated (B-D). Messenger RNA target independent STIC complexes seen as much brighter puncta are indicated in 1A by arrowhead. Bottom row; Cy3 (orange), DAPI (blue) and DIC (grey) merged images of cells above it. Non-hybridizing STIC complexes are indicated with white arrowheads in panel A. Scale bars: 10 μm.

FIG. 3. Simultaneous detection of Actb and Actg mRNAs with in situ hybridization probes. Two Actb and two Actg primary nucleic acid elements were synthesized and corresponding secondary and tertiary nucleic acid elements were synthesized to label Actb mRNA with Cy3 and Actg mRNA with Cy5 probes. In situ hybridization probes were hybridized to primary MEF cells and imaged with epifluorescence microscope. Top row: Normalized Cy3 images of cells after hybridization with complete FISH probes (A and B) or in situ hybridization probes omitting one set of primary nucleic acid elements as indicated (C and D). Middle row: Normalized Cy5 images of cells after hybridization with complete FISH probes (A′ and B′) or in situ hybridization probes omitting one set of primary nucleic acid elements as indicated (C′ and D′). Bottom Row; Cy3 (red), Cy5 (green) and DAPI (blue) merged images of cells above it. Panel B, B′ and B″ are detailed images of the boxed regions of interest (ROI) in panels A, A′ and A″ respectively. Scale bars for columns A, C and D: 10 μm. Scale bar for column B: 2 μm.

FIG. 4. in situ hybridization probes probe specificity. Two Actb and three mouse choline acetyl-transferase (ChAT) primary nucleic acid elements were synthesized to label Actb mRNA with Cy3 and simultaneously label ChAT mRNA with Cy5. Probes were hybridized to primary MEF cells and imaged by epifluorescent microscopy. Top row: Images of FITC channel autofluorescence images (A), Cy3 (B) and Cy5 (C) from a single cell. Middle row: A′ B′ and C′ correspond to and expanded view of the ROI indicated by the dashed line box in images A, B and C respectively. Bottom Row: Normalized images of FITC autofluorescence (D), Cy3 (E) and Cy5 (F) taken from one cell hybridized without a secondary nucleic acid elements as an imaging control. A-F are maximum projection images of Z-series of corresponding images. Scale bars: 10 μm.

FIG. 5. Actb and Actg have spatially distinct distribution in the same cells. Two Actb and two Actg primary nucleic acid elements were hybridized to primary MEF cells and imaged by epifluorescent microscopy. Representative normalized Cy3 images for Actb or Cy5 for Actg are shown in panels A and B, respectively. The merged images are shown in panel C, with the ROI indicated by the dashed box in C shown in panel D. Control images from hybridizations without secondary nucleic acid are shown for Cy3 (E) and Cy5 (F). The Images shown are deconvoluted from Z series taken at 60×. Polarization index (G) and Distribution index (H) for 78 images are represented as box-whisker plots, with the median (black line) and middle quartiles represented in the box, the highest and lowest quartiles represented in the whiskers, and outliers indicated by circles. Polarization and Distribution indexes were calculated from maximum projection images of non-deconvolved Z-series using a Mann-Whitney Rank Sum Test. Scale bars: 10 μm, except panel D, scale bar: 2 μm.

FIG. 6. in situ hybridization probes detection of Nrg1-III and Actg mRNA in primary neurons. Embryonic day 18 cortical neurons were plated on poly-lysine coated coverslips and maintained in culture for 10 Days in vitro (DIV). 5-Fluoro-deoxyuridine (FDU) was added after 3 DIV. Neurons were fixed, and then co-hybridized with Cy3 Nrg1-III (A), Cy5 Actg (B) primary nucleic acid elements and corresponding secondary nucleic acid elements. Normalized images from control hybridization reactions lacking any secondary nucleic acid molecules are shown in panels D and E. DIC images of the cells imaged in Cy3 and Cy5 are shown in panel C and panel F of the hybridizations indicated. Images are single plane epifluorescence taken at 60× magnification. Scale bar: 10 μm.

FIG. 7. Design and use of coverslip dislodging tool for use in the current methods. A) Shows the modification of an 18-gauge syringe needle into a coverslip dislodging tool. B) Depicts the use of a coverslip dislodging tool in the current methods, wherein the coverslip dislodging tool slides between the side of the culture dish well and the side of the coverslip (black oval) while buffer is in the well (dotted line shows the top of the buffer line). The tip of the dislodging tool is slid under a portion of the coverslip, keeping the tip in contact with the bottom of the well. Then the tip of the dislodging tool is used to gently lift and slide the coverslip to the opposite side of the well while the lifted edge of the coverslip rests on the shaft of the too. The coverslip will lift up in a manner that permits the coverslip to be grasped by forceps or pliers. C) Clearly shows how the dislodging tool is used to lift and remove a coverslip from a humidified chamber. Here, a front most surface (i.e., tip) of a forcep is placed immediately adjacent to parafilm abutting a coverslip in order to prevent the coverslip from sliding as the dislodging tool is used to gently dislodge and lift the opposite end of the coverslip. The tool is then moved toward the forceps allowing the lifted edge of the coverslip to rest on the shaft of the dislodging tool. Ultimately, the angle of the coverslip will be steep enough that it will stand up on its own while resting on the tool, then the forceps can be used to grasp and remove the coverslip.

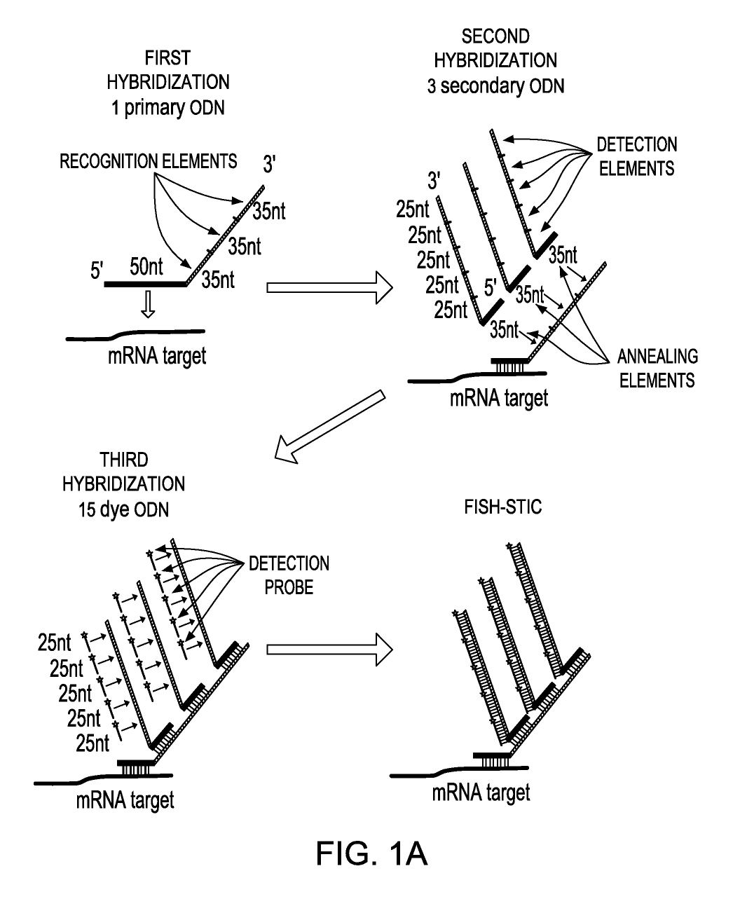

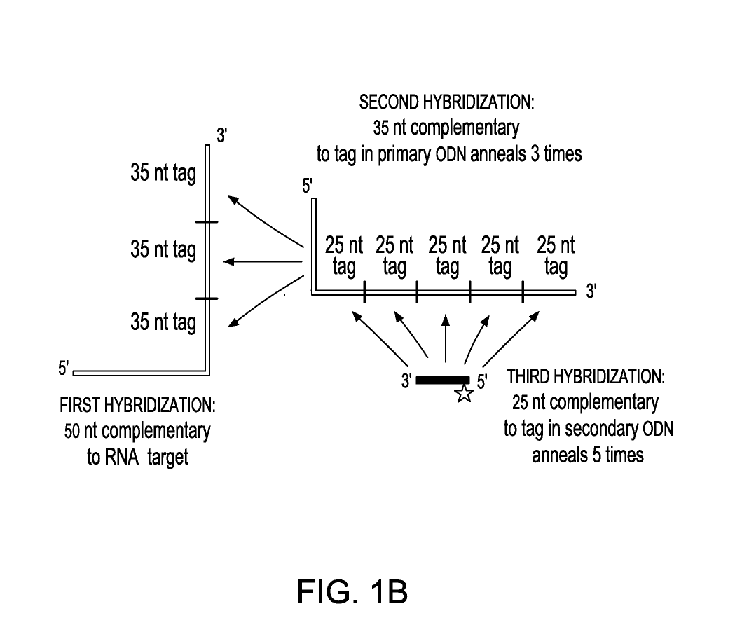

Table 1. Oligonucleotides used in experimental examples. Nucleic acid element sequences are written 5′ to 3′. Transitions between individual sequence features of each nucleic acid, as described in FIGS. 1A-B, are indicated by a change from capital to lowercase, or vice versa.

Table 2. Concentrations of reagents used for development of solutions used in the current methods. Amounts of materials used to develop 50 μl of primary, secondary and tertiary nucleic acid element solutions for use in the current FISH-STIC methods.

### DETAILED DESCRIPTION OF THE DISCLOSURE

### Definitions

Unless defined otherwise, all technical and scientific terms used herein have the same meaning as commonly understood by one of ordinary skill in the art to which the current disclosure pertains. The following definitions supplement those in the art and are directed to the current disclosure. Accordingly, the terminology used herein is for the purpose of describing particular embodiments only, and is not intended to be limiting.

The term “nucleic acid” or “oligonucleotide” or “ODN” encompasses any physical string or collection of monomer units (e.g., nucleotides) that can connect to form a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the nucleic acid can be deoxyribonucleotides, ribonucleotides or nucleotide analogs, and can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The nucleic acid can additionally comprise non-nucleotide elements such as labels, quenchers, blocking groups, or the like. The nucleic acid can be single-stranded or double-stranded.

The term “nucleic acid of interest”, “target nucleic acid”, “target DNA” or “target RNA” as used herein includes a nucleic acid originating from one or more biological entities within a sample. Wherein a biological entity as used herein means any independent organism or thing, alive or dead, containing genetic material (e.g., nucleic acid) that is capable of replicating either alone or with the assistance of another organism or cell. Non-limiting examples of sources for nucleic acid containing biological entities of the current disclosure include an organism or organisms including, a cell or cells, bacteria (e.g., Gram positive or Gram negative), yeast, fungi, algae, viruses, or a sample thereof. Specifically, an organism of the current disclosure includes bacteria, algae, viruses, fungi, and mammals (e.g., humans). Wherein the term “sample”, refers to a portion of a larger material such as a biological entity containing nucleic acid(s). Specifically, the sample contains genetic material including, nucleic acids processed or isolated from a biological entity by methods known to one of ordinary skill in the art. Non-limiting examples of isolation techniques include nucleic acid extraction or isolation methods, nucleic acid amplification, surgical resection of a tissue and the like.

A “nucleotide sequence” or “nucleic acid sequence” is a polymer of nucleotides (an oligonucleotide, a DNA, a nucleic acid) or a character string representing a nucleotide polymer, depending on context. From any specified nucleic acid sequence, either the given nucleic acid or the complementary nucleotide sequence (e.g., the complementary nucleic acid) can be determined.

The term “element” or “elements” as used in the current disclosure means a moiety that is a portion of a larger moiety, composition or method. In certain embodiments a primary nucleic acid molecule may be an element of a larger complex of nucleic acids. In yet another embodiment primary, secondary, tertiary nucleic acid moieties and a probe are elements of the in situ hybridization probes of the current disclosure.

The term “binding”, “to bind”, “binds”, “bound” or any derivation thereof refers to any stable, rather than transient, chemical bond between two or more molecules, including, but not limited to, covalent bonding, ionic bonding, and hydrogen bonding. Thus, this term also encompasses interaction between a nucleic acid molecule and another entity such as, a nucleic acid or probe element. Specifically, binding, in certain embodiments, includes the hybridization of nucleic acids.

The term “hybridize” or “hybridization” as used in the instant disclosure shall mean the association of two nucleic acids to form a stable duplex. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.).

The term “complementary” refers to a nucleic acid that forms a stable duplex with its “complement”. For example, nucleotide sequences that are complementary to each other have mismatches at less than 20% of the bases, at less than about 10% of the bases, preferably at less than about 5% of the bases, and more preferably have no mismatches.

A “recognition element” is a nucleotide sequence that is capable of hybridizing to annealing element. In certain embodiments the recognition element is a portion of a first nucleic acid element of an FISH-STIC molecule or oligonucleotide having a nucleotide sequence, which is complementary to the nucleotide sequence of the annealing element, typically present on a second nucleic acid element or oligonucleotide (“secondary nucleic acid element”). In certain embodiments the recognition element is single-stranded. In certain embodiments the recognition element is less than or equal to 50 nucleotides in length, less than or equal to 40 nucleotides in length, or less than or equal to 35 nucleotides in length. In other embodiments the recognition element is between 10 and 50 nucleotides in length, inclusive. In yet another embodiment the recognition element is between 20 and 40 nucleotides in length, inclusive. In other embodiments the recognition element is between 30 and 40 nucleotides or 30 and 35 nucleotides in length, inclusive.

The term “annealing element” means a nucleotide sequence complimentary to the nucleotide sequence of at least one recognition element and is capable of binding to any recognition element. In one embodiment the annealing element is a portion of a second nucleic acid element or oligonucleotide having a nucleotide sequence, which is complementary to the nucleotide sequence of a recognition element. In certain embodiments the annealing element is less than or equal to 50 nucleotides in length, less than or equal to 40 nucleotides in length, or less than or equal to 35 nucleotides in length. In one embodiment the annealing element is between 10 and 50 nucleotides in length, inclusive. In yet another embodiment the annealing element is between 20 and 40 nucleotides in length, inclusive. In other embodiments the recognition element is between 30 and 40 nucleotides or 30 and 35 nucleotides in length, inclusive. In one embodiment the annealing element is the same length as the recognition element.

The term “detection element” as used in the instant disclosure means a nucleic acid capable of hybridizing to at least one tertiary nucleic acid element or a detection probe. In certain embodiments the detection element is a portion of a second nucleic acid or oligonucleotide having a nucleotide sequence, which is complementary to the nucleotide sequence of the third nucleic acid or oligonucleotide containing or bound to a detection probe. In certain embodiments the detection element is single-stranded. In one embodiment the detection element is hybridized to a third nucleic acid or oligonucleotide containing or bound to a detection probe, covalently or non-covalently, directly or through a linker (e.g., streptavidin-biotin). In certain embodiments the detection element is less than or equal to 50 nucleotides in length, less than or equal to 40 nucleotides in length, or less than or equal to 35 nucleotides in length. In other embodiments the detection element is between 10 and 50 nucleotides in length, inclusive. In yet another embodiment the detection element is between 20 and 40 nucleotides in length, inclusive. In other embodiments the detection element is between 10 and 30 nucleotides or 15 and 25 nucleotides in length, inclusive. In one embodiment the detection element contains the same number of nucleotides as a third nucleic acid or oligonucleotide containing or bound to a detection probe.

A “detection probe” or “probe” is a moiety that facilitates detection of a molecule present on or otherwise connected to a tertiary nucleic acid element. Non-limiting examples of detection probes for use in the current disclosure include fluorescent, luminescent, light-scattering, and/or colorimetric molecules. Suitable detection probes include, but are not limited to enzymes and fluorescent moieties, as well as radionucleotides, substrates, cofactors, inhibitors, chemiluminescent moieties, magnetic particles, antibodies and other molecular probes commonly known to the skilled artisan.

The term “primary nucleic acid element” of a FISH-STIC probe of the instant disclosure shall mean a nucleotide sequence, which is complementary in sequence to a nucleic acid of interest or endogenous target nucleic acid sequence present in an environmental sample, biological entity or sample thereof and includes at least one recognition element that sequentially succeeds the portion of the primary nucleic acid containing a nucleotide sequence that is complementary to a nucleic acid of interest.

The term “secondary nucleic acid” of a FISH-STIC probe of the instant disclosure shall mean a nucleotide sequence, which includes at least one annealing element containing a nucleotide sequence, present at the 5′ end of the secondary nucleic acid element that is capable of hybridizing to any one of the recognition elements in a primary nucleic acid element, and at least one detection element that sequentially succeeds the annealing element(s) present at the 5′ end of the secondary nucleic acid element.

A “tertiary nucleic acid element” as used herein is an oligonucleotide that includes at least one nucleic acid sequence that is capable of hybridizing to any one of the detection elements of the secondary nucleic acid elements of the in situ hybridization probe and at least one detection probe that directly or indirectly provides a detectable signal. In certain embodiments the nucleic acid sequence connected to the detection probe is less than or equal to 50 nucleotides in length, less than or equal to 40 nucleotides in length, or less than or equal to 35 nucleotides in length. In other embodiments the nucleic acid sequence connected to the detection probe is between 10 and 50 nucleotides in length, inclusive. In yet another embodiment the nucleic acid sequence connected to the detection probe is between 10 and 25 nucleotides in length, inclusive. In other embodiments the nucleic acid connected to a detection probe is between 10 and 20 nucleotides or 15 and 25 nucleotides in length, inclusive. In one embodiment the nucleic acid connected to the detection probe contains the same number of nucleotides present in a detection element.

### In Situ Hybridization Probes

The ability to detect nucleic acid molecules in situ has long had important applications for molecular biological studies. Enzyme or dye-labeled antisense in vitro runoff transcripts or synthetic oligodeoxynucleotides both have a proven track record of success, but each of these also has scientific and practical drawbacks and limitations to their use. For example, nucleotide expression has traditionally been measured using Northern blot and nuclease protection assays. However, these approaches are time-consuming and have limited sensitivity. Greater sensitivity and quantification is possible with reverse transcription polymerase chain reaction (RT-PCR) based methods, such as quantitative real-time RT-PCR, but these approaches have low multiplex capabilities. Additionally, presently available in situ hybridization probes are synthesized in the 3′ to 5′ direction, increasing the probability of premature truncation of the probes during synthesis, a deficiency currently overcome by the methods and compositions of the current disclosure.

The compositions and methods of the current disclosure employ oligonucleotides as nucleic acid-FISH probes for detection of target nucleic acids (e.g., RNA or DNA). The current compositions and methods facilitate the binding of a high concentration of detection probes to a stretch of nucleotides using, for example, commercial DNA synthesis outlets available to any lab.

The in situ hybridization probes of the current disclosure place a complementary hybridizing nucleotide sequence at the 5′ end of the respective nucleic acid element, thus any truncated or improperly formed nucleic acids will be unable to hybridize. This novel characteristic prevents binding of truncated or improperly formed nucleic acid probe elements, and thus limits the number of false positives detected by the probes.

The in situ hybridization probes of the current disclosure include a primary (first), at least one secondary (second) and at least one tertiary (third) nucleic acid element each consisting of between 5 and 200 nucleotides, 5 and 155, 5 and 100, 10 and 75 nucleotides, 5 and 50, or 10 and 50 nucleotides, inclusive.

One aspect of the current disclosure includes in situ hybridization probes to detect the relative amounts of at least one target nucleic acid in a sample or plurality of samples by detecting a target nucleic acid with in situ hybridization probes labeled with a diagnostic probe unique to each of the target nucleic acid sequences being identified, and detecting the hybridization of each of the corresponding in situ hybridization probes to its respective target nucleic acid.

In one embodiment a primary nucleic acid element includes a nucleotide sequence of about 50 nucleotides, which is complementary in sequence to a nucleic acid of interest or target nucleic acid present in a biological entity or sample thereof. In certain embodiments the biological entity is a tissue, or collection of cells. In yet another embodiment the biological entity is blood, or plasma. In yet another embodiment the biological entity is a bodily fluid, such as urine or spinal fluid. In one embodiment the biological entity is bacteria.

In yet another embodiment the nucleic acid sequence located at the 5′ end of the primary nucleic acid element and complementary to the nucleic acid sequence of interest or target nucleic acid is of any size, including, but not limited to, 10 to 80, 15 to 70, 20 to 60, 25 to 55, 35 to 50 or 40 to 50 nucleotides in length, inclusive. In one embodiment the nucleotide sequence of about 50 nucleotides that are complementary to a nucleic acid of interest is located at the 5′ end of the primary nucleic acid. In yet another embodiment the primary nucleic acid element contains a nucleic acid sequence of 55 nucleotides.

In certain embodiments the primary nucleic acid element includes about 3 identical recognition elements that sequentially succeed the portion of the primary nucleic acid containing a nucleotide sequence that is complementary to a nucleic acid of interest. In certain embodiments the recognition elements are synthesized from 5′ to 3′ to prevent against the development of premature truncation and improper hybridization of the recognition elements. In one embodiment the nucleotide sequence of the recognition element(s) is randomly generated through a random sequence generating website then screened by BLAST search to limit background complementarity, and thus creating a sequence lacking a high complementarity to existing nucleic acids present in a biological entity.

In certain embodiments the number of recognition elements present in a first nucleic acid is between 2 and 10, 2 and 7, 2 and 5 or 2 and 4. In certain embodiments the number of recognition elements present in the first nucleic acid element is 3, 4 or 5.

In other embodiments, the recognition elements are of any size, including, but not limited to, 5 to 50, 10 to 45, 20 to 40, 25 to 40, 30 to 40 and 30 to 35 nucleotides in length, inclusive. In certain embodiments the recognition elements are about 35 nucleotides in length.

In certain aspects of the current disclosure the in situ hybridization probes contain at least one secondary nucleic acid element. In one embodiment the number of secondary nucleic acid elements is between 1 and 10, 2 and 7, 2 and 5 or 2 and 4. In certain embodiments the number of secondary nucleic acid elements present is 3, 4 or 5. In yet another embodiment the number of secondary nucleic acid elements present is identical to the number of recognition elements present in the primary nucleic acid element.

In certain embodiments the secondary nucleic acid element of the current disclosure includes at least one annealing element, present at the 5′ end of the secondary nucleic acid. In certain embodiments the nucleic acid sequence of the annealing element is capable of hybridizing to any one of the recognition elements in the primary nucleic acid element. In other embodiments the sequence of the annealing element is complementary to that of the recognition element(s) present in a primary nucleic acid element. In certain embodiments the annealing element(s), which is located at the 5′ end of the secondary nucleic acid element is between 5 and 50, 10 and 45, 20 and 40, 25 and 40, and 30 and 40 nucleotides in length, inclusive. In certain embodiments the annealing element(s) are about 35 nucleotides in length. In yet another embodiment the annealing element(s) are identical in size to the recognition elements present in a primary nucleic acid element.

In certain embodiments the secondary nucleic acid elements of the current disclosure include about 5 detection elements that sequentially succeed the annealing element(s) present at the 5′ end of the secondary nucleic acid element(s). In other aspects of the current disclosure each detection element contains a nucleotide sequence of about 20 to 25 nucleotides in length. In certain embodiments the detection elements are synthesized from 5′ to 3′ to prevent against the development of premature truncation and improper hybridization to a tertiary nucleic acid element of the current disclosure. In other embodiments, the detection elements are of any size including, but not limited to, 5 to 50, 10 to 45, 15 to 35, 20 to 30, and 20 to 25 nucleotides in length, inclusive. In certain embodiments the detection elements are about 25 nucleotides in length.

In one embodiment the nucleotide sequence of the detection element(s) is randomly generated through a random sequence generating website then screened by BLAST search to limit background complementarity, thus creating a sequence lacking a high complementarity to existing nucleic acids present in an environmental sample, biological entity, sample or other elements of the in situ hybridization probe. In other embodiments the nucleotide sequence of the detection element(s) are complementary to that of a tertiary nucleic acid element containing a probe. In other aspects of the instant disclosure the nucleotide sequence of detection elements present in the secondary nucleic acid elements are capable of hybridizing to that of a tertiary nucleic acid element containing a probe.

In certain embodiments the number of detection elements present in a secondary nucleic acid element is between 2 and 10, 2 and 7, 2 and 5 or 2 and 4. In certain embodiments the number of detection elements present in a second nucleic acid is 3, 4, 5 or 6.

In certain aspects of the current disclosure the in situ hybridization probes contain at least one tertiary nucleic acid element. In certain embodiments the number of tertiary nucleic acid elements is between 1 and 50, 1 and 30, 5 and 25, 1 and 15, or 3 and 15. In other embodiments the number of tertiary nucleic acid elements present is 3, 6, 9, 12, or 15. In yet another embodiment the number of tertiary nucleic acid elements is identical to the number of detection elements present in the secondary nucleic acid element(s).

In certain embodiments the tertiary nucleic acid elements of the current disclosure includes a nucleotide sequence of about 20 to 25 nucleotides. In certain embodiments, the tertiary nucleic acid elements are of any size, including, but not limited to, 5 to 50, 10 to 45, 15 to 35, 20 to 30, and 20 to 25 nucleotides in length, inclusive. In certain embodiments the detection elements are about 25 nucleotides in length.

In certain embodiments the nucleic acid sequence of a tertiary nucleic acid element is capable of hybridizing to any one of the detection elements of a secondary nucleic acid element of the in situ hybridization probe. In other embodiments the nucleic acid sequence of the tertiary nucleic acid element is complementary to that of the detection element(s) present in a secondary nucleic acid element.

In a specific embodiment of the current disclosure, a tertiary nucleic acid element includes at least one detection probe. Detection probe(s) can be located at any point of the tertiary nucleic acid element including but not limited to the 3′end or 5′end of the tertiary nucleic acid element. Suitable detection probes include, but are not limited to, enzymes and fluorescent moieties, as well as radionucleotides, substrates, cofactors, inhibitors, chemiluminescent moieties, magnetic particles, antibodies and other molecular probes commonly known to the skilled artisan.

In certain embodiments the detection probe is hybridized or otherwise connected to a nucleic acid capable of hybridizing to the detection element present in or on a second amino acid or oligonucleotide (e.g., a single-stranded nucleic acid including a probe or that is configured to bind to a probe) that directly or indirectly provides a detectable signal. In an embodiment the detection probe is connected to a nucleic acid that is complementary to a detection element.

### Methods of Synthesizing In Situ Hybridization Probes

The methods of developing the in situ hybridization probes of the instant application includes the sequential hybridization of at least three nucleic acid elements, of decreasing length to facilitate stringent hybridization, and thus limit improper hybridization (e.g., hybridization to endogenous nucleic acid molecules).

Hybridization generally includes the process of combining (e.g., binding) two complementary single-stranded DNA or RNA nucleic acid elements. Nucleic acid hybridization enables the formation of a single double-stranded molecule through the interaction of complementary nucleotide bases. More specifically, a hybridization probe (e.g., in situ hybridization probe) contains a nucleic acid sequence that is complementary to a target nucleotide sequence, which is bound to a detection probe (e.g., radiolabel or fluorescent molecular marker) facilitates the visualization of the target nucleic acid sequence.

Generally, nucleic acid hybridization occurs under stringent conditions, whereby the temperature at which a nucleic acid molecule is elevated in the presence of a concentrated salt buffer (e.g., hybridization solution or salt buffer), which facilitates the binding of complementary nucleotides. In instances whereby nucleotide sequences to be hybridized contains a reduced (i.e., less than 50%) G-C content, less stringent conditions, e.g., lower heat and increased salt concentration will also permit the hybridization of nucleic acid sequences with less complementarity.

One aspect of the current disclosure includes a method for manufacturing in situ hybridization probes for the detection of target nucleic acid sequences including, for example; (1) selecting at least one nucleic acid of interest present in a sample; (2) designing a primary nucleic acid element, which includes a nucleotide sequence located at the 5′end of the primary nucleic acid element that is complimentary to a portion of the nucleic acid of interest and capable of hybridizing to such nucleic acid of interest, wherein the primary nucleic acid element also includes at least one recognition element; (3) designing a secondary nucleic acid element, that includes at least one annealing element located at the 5′ end of the second nucleic acid element that is complimentary to the nucleotide sequence of the of recognition elements present in the primary nucleic acid element, and a plurality of detection elements; (4) designing a tertiary nucleic acid element, that includes a nucleotide sequence complementary to the nucleic acid sequence of the detection element(s) present in the secondary nucleic acid element, and a detection probe; (5) the primary, secondary and tertiary nucleic acid elements are then sequentially hybridized to the nucleic acid of interest. The sequential hybridization step can be carried out under conditions that prevent dissociation of previously hybridized nucleic acid elements, which are well known in the art.

Any target nucleic acid sequences of interest may be selected for use in the method of the current disclosure. Suitable target nucleic acid sequences include any type of nucleic acid, for example, DNA or RNA, or a nucleic acid mimic, or a combination thereof. In one embodiment, the target nucleic acid sequences can include messenger RNAs or RNA transcripts. In another embodiment of the method, a target nucleic acid sequence can include genomic DNA. In yet another embodiment of the method, a target nucleic acid sequence can include nucleic acid constructs such as plasmids optionally including inserted sequences. In yet another embodiment a nucleic acid of interest may be a non-coding RNA including, but not limited to, microRNA.

In one embodiment the methods of the current disclosure include, for example, the design of a primary nucleic acid directed against 50 nucleotides of an RNA or DNA molecule of interest; a secondary nucleic acid element that hybridizes to a trimerized unique recognition element present at the 3′ end of the primary nucleic acid element; and a tertiary nucleic acid element that hybridizes to the secondary nucleic acid element by contacting a detection element located at the 3′ end of the secondary nucleic acid element. In certain embodiments, the tertiary nucleic acid element is directly coupled to a fluorescent dye or probe that enables detection of the nucleotide of interest through the sequential hybridization of in situ hybridization probes.

The methods of the present disclosure can also be manipulated to create in situ hybridization probes useful in detecting at least two target nucleic acids.

Generally, nucleic acid probes are detected by hybridization with labeled nucleic acid sequences complementary to a region within a nucleic acid of interest nucleic acid (i.e., target nucleic acid). Non-limiting examples of probe detection techniques include microscopy and flow cytometry. Flow cytometry, involves analyzing cells or fractions thereof that are suspended in a solution and stained with fluorescent dyes (e.g., Cy3, Cy5, Cy7). In flow cytometry, cells are directed into a narrow stream whereby they are exposed to a laser light. As the cells pass the laser beam in they are in a single file, and thus when contacted with the laser light beam individual cells scatter light or emit fluorescence. As each cell passes through the light source, its optical properties are quantified, processed and stored using computer programs known to one of ordinary skill in the art.

In one non-limiting embodiment of the current disclosure a probe can be incorporated during synthesis of the tertiary nucleic acid element, for example, by incorporation of radioactively labeled bases such as 35S-dNTP, 32P-dNTP, 33P-dNTP, 14C-dNTP, or by incorporation of non-radioactively labeled bases such as, but not limited to, digoxin- or digoxygenin-labeled dNTP, biotin-labeled dNTP, fluorophore-labeled dNTP, or hapten-labeled dNTP. In one non-limiting example, oligonucleotide probes intended for use in in situ hybridization can be internally labeled during synthesis using a modified amino-allyl-dT.

In certain embodiments, the tertiary nucleic acid element is first synthesized from unlabelled bases or, alternatively, with bases bearing functional groups to which a detectable label can be later attached, and the probe is attached to the tertiary nucleic acid element, directly or indirectly, by covalent or non-covalent means or a combination thereof, after the nucleic acid is synthesized.

In certain embodiments, the probe may be attached to the tertiary nucleic acid element after hybridization occurs. For example, a biotinylated probe may hybridize to its nucleic acid sequence complement, and be detected using an avidin-labelled enzyme and an appropriate enzyme substrate. Methods to introduce such functional groups or detectable labels are known in the art. See R. P. Haugland, “Handbook of Fluorescent Probes and Research Products”, 9^{th} edition, J. Gregory (editor), Molecular Probes, Inc., Eugene, Oreg., USA, 2002.

The primary, secondary and tertiary nucleic acid elements of the current methods may be made by any technique suitable to the composition of the particular nucleic acid. For example, a nucleic acid element may include only a nucleic acid (DNA or RNA) and such an oligonucleotide probe may be made by any suitable DNA, RNA. See, J. Sambrook and D. Russell, “Molecular Cloning: A Laboratory Manual”, third edition (2001), Cold Spring Harbor Laboratory Press, New York, 2.

In a specific embodiment of the present disclosure three nucleic acid elements are used: (i) a 155 nucleotide primary nucleic acid element; (ii) a 160 nucleotide secondary nucleic acid element; and (iii) a 25 nucleotide tertiary (dye) nucleic acid element as shown in FIG. 1B. In certain instances the primary and secondary nucleic acid elements are synthesized at amounts of about 4 nmol and resuspended in water to a 50 μM final concentration, while the tertiary (dye) nucleotide element is resuspended to a concentration of 100 μM in H₂O for long-term storage. In certain embodiments the nucleic acid elements developed herein can be analyzed by polyacrylamide gel electrophoresis (PAGE) to ensure they are the correct length. The methods further include the development of hybridization solutions whereby the 50 μM solutions of primary or secondary nucleic acid elements are further diluted into 5 μM aliquots.

During primary nucleic acid element design the 5′ end of the oligomer contains 45-55 nucleotides that are complimentary to the endogenous target molecule of interest (FIG. 1B). In certain specific embodiments the hybridization site of the primary nucleic acid element is be between 40 and 60% G-C content. However, higher and lower G-C can be used by forming shorter regions of complementarity when G-C content is greater than 60% or by decreasing formamide concentration in the primary hybridization solution when G-C content is less than 40% of the nucleotides in the primary nucleic acid element. In certain embodiments BLAST searchers or NCBI databases are used to select and confirm appropriate stretches of a target sequence. In yet another embodiment of the present disclosure a BLAST search is used to confirm that the nucleotide sequence of the primary nucleic acid element is not present in any nucleotide containing molecule of the target organism or sample.

In a specific embodiment a 35 nucleotide sequence for a recognition element of a primary nucleic acid element with a 50% G-C content is synthesized using a random nucleotide sequence generator that provides a 50% GC content nucleotide sequence. This sequence is then used in a BLAST search to ensure the junction between the portion of the primary nucleic acid element that is complementary to the target nucleotide sequence and the recognition element does not create a stable off target hybridization. When a suitable nucleotide sequence for a recognition element is identified three identical copies of the recognition element are appended to the 3′ end of the portion of the primary nucleic acid element that is complementary to the target nucleotide sequence.

In certain instances, wherein the target nucleic acid molecule is greater than 100 nucleotides in length, multiple primary nucleic acid elements can be designed and utilized to target different stretches of the target nucleic acid molecule, which include the same recognition element(s). In this instance all of the recognition element sequences can be concurrently hybridized by including 5 μM of each of individual primary nucleic acid element in the hybridization solution, as described below.

During secondary nucleic acid element synthesis the 5′ end of a recognition element of the primary nucleic acid element is anti-parallel to an annealing element of a secondary nucleic acid element (FIG. 1B). Next, a detection element with a random sequence having about a 50% G-C content is designed using a random nucleotide sequence generator, and checked to ensure that no off target hybridization will occur. Once the detection element sequence is determined, at least 5 copies of the detection element are synthesized and appended to the 3′ end of the annealing element of each secondary nucleic acid element.

A tertiary nucleic acid element is synthesized so that a nucleotide sequence that is complementary to that of a detection element on a secondary nucleic acid element is anti-parallel to a detection element of a secondary nucleic acid element, and includes a detection probe (e.g., fluorophore) on the 5′ end (FIG. 1B). In certain embodiments, other detection elements (e.g., Cy3, Cy5, radiolabels) are incorporated into the tertiary nucleic acid element to facilitate detection of the target sequence. FITC and similar spectrum fluorophores coincide with the highest cellular autofluorescence so should be avoided for use as detection agents for incorporation of the tertiary nucleic acid element avoid using these as fluorophores for FISH.

In certain embodiments, the nucleic acid elements may be synthesized without additional purification procedures, as experimental results have revealed that nucleic acid elements synthesized without additional purification steps have not exhibited a reduction in nucleic acid element quality (data not shown). However, the tertiary nucleic acid element, which is synthesized to include a 5′ end is labeled with a detection probe, is preferably purified to increase labeling efficiency.

### Methods of Using In Situ Hybridization Probes

In Situ Hybridization is a molecular technique that permits the precise localization and detection of a specific nucleic acid within an organism or sample thereof. The underlying basis of in situ hybridization is that nucleic acids can be targeted and detected through the application of a complementary strand of nucleic acid to which a detection probe is attached. Hybridization and detection of the detection probe enables the visualization of an endogenous target, such as nucleic acid sequences in an organism or a sample thereof, an environmental sample, or a cell

In one aspect of the instant disclosure the in situ hybridization compositions provided herein are used in conjunction with various in situ hybridization techniques including, but not limited to fluorescent in situ hybridization, chromogenic in situ hybridization, in cells, tissue sample preparations, organisms or environmental samples containing nucleic acids.

In one embodiment of the present disclosure the in situ hybridization compositions of the instant disclosure are used in a fluorescent in situ hybridization assay to detect target nucleic acids, such as RNA or DNA. In a specific embodiment, parafilm is spread in the bottom of a cell culture dish and in situ hybridization probe solution is placed on the parafilm as an array of aliquots of solution in an alignment that prevents coverslips placed on the parafilm from contacting each other during incubation. Coverslips are then placed onto each aliquot of primary nucleic acid element solution. Next, phosphate buffer solution is added to each coverslip to maintain humidity of the culture during incubation. The lid on the culture dish is then sealed to facilitate cell growth on the coverslips.

Coverslips are then recovered and the cells are fixed (e.g., using formaldehyde or paraformaldehyde fixation techniques known to one of ordinary skill in the art). Coverslips containing fixed cells are then incubated in methanol, and serially rehydrated with 40% formamide, followed by one complete solution change into fresh 40% formamide. Coverslips are then incubated with primary nucleic acid elements (prepared as above) cells-side down in a humidified chamber and incubated overnight.

The coverslips are then removed from the parafilm and individually placed cells-side up into separate wells of a culture dish with a 40% formamide and incubated. In certain embodiments the incubation with formamide is repeated to equilibrate the coverslips prior to secondary nucleic acid element hybridization.

Coverslips are then placed cells-side down on a drop of secondary nucleic acid element mixture in a hybridization chamber. The chamber is then sealed and the cells are incubated. The coverslips are then gently pried off of the parafilm, individually placed cells-side up into a culture dish with 35% formamide and incubated. In certain embodiments the incubation with formamide is repeated to equilibrate the cover-slips for the tertiary nucleic acid element hybridization.

Coverslips are then placed cells-side down on a drop of tertiary nucleic acid element mixture in a hybridization chamber. The chamber is then sealed and incubated in darkness. The coverslips are then removed from the parafilm and individually placed cells-side up into separate wells of a culture dish with 20% formamide and then incubated. In certain embodiments the incubation with formamide is repeated then the buffer changed to 1×SSC, with a detergent (e.g., Tween 20 or TritonX) and DAPI and incubated. Cells are then rinsed at least once in SSC and mounted in microscopy mounting medium, and visualized using microscopy.

### EXAMPLES

The following examples further illustrate the disclosure, but should not be construed to limit the scope of the disclosure in any way.

### Example 1

### Method of Creating ODN Probes. Primer Design

Primary (i.e., first) nucleic acid elements include a 45 to 50 nucleic acid sequence antisense to a gene specific mRNA at the 5′ end of a DNA nucleic acid, followed by 3 copies of a 35 nucleotide recognition element. Multiple primary nucleic acid elements, which are complementary to the same mRNA were designed with non-overlapping 50 nucleotide nucleic acid sequences complementary to different sequences within the same mRNA transcript but containing the same 35 nucleotide recognition element. The secondary nucleic acid element(s) contain a 35 nucleotide annealing element that is complementary to the 35 nucleotide sequence of the recognition element in a primary nucleic acid, followed by 5 copies of a random 25 nucleotide detection element. The tertiary nucleic acid element (i.e., third) or dye ODN is a 25 nucleotide sequence that is complementary to the 25 nucleotide sequence in a detection element of the secondary nucleic acid element and contains a fluorescent dye at the 5′ end (Cy3 for the Actb probes and Cy5 for the Actg probes). All DNA sequences were targeted to consist of 50% G-C base pairs, with actual ratios varying between 40% and 60%. Individual antisense 50 nucleotide nucleic acids and randomly generated 35 nucleotide nucleic acid sequences and 25 nucleotide sequences were subjected to BLAST search to minimize the potential to cross-hybridize to other mRNA sequences in the genome. Typically 14-16 continuous bases were the largest stretch of complementary sequence found in potential off-target sequences. Primary and secondary nucleic acid elements were synthesized as standard-desalted Ultramers, and tertiary nucleic acid elements were created with the indicated dye at the 5′end and purified.

Cell Culture.

Primary mouse embryonic fibroblasts (MEFs) were isolated from e14 mouse embryos by standard procedures and immortalized by transfection with SV40 middle T antigen expressing plasmid. See Lu P D, et al. EMBO J (2004) 23: pp. 169-179. Cells were maintained in DMEM with 10% FBS with 10 μg/ml gentamicin (D10). MEFs were plated at a density of 25,000 cells on coated 18 mM coverslips in a 12 well culture dish in D10. The coverslips were coated using 50 μg/ml poly-1-lysine in boric acid buffer (50 mM boric acid, 5 mM sodium tetraborate, pH 8.5) over-night at room temperature, then washed in sterile water prior to adding cells. The cells were allowed to attach and grow over-night before being fixed using four two-fold dilutions of 4% paraformaldehyde with 1 mM MgSO₄. Cells were allowed to fix in the final dilution for 20 minutes. The cells were then washed in PBS with 0.1M glycine (PBSG) for 10 minutes and then permeabilized and stored in 80% methanol at −20° C. overnighNeuronal cultures were maintained according to the procedure described previously. See Sinnamon J R, et al. RNA (2012) 18 pp. 704-719. Briefly, embryonic day 18 timed pregnant mice were sacrificed using CO₂ in accordance with IACUC protocols. Cortices were isolated from the pups, trypsinized, dissociated and plated in neurobasal supplemented with B27, primocin, and glutamax. After three days in vitro cultures were treated with 3 μM FDU. Following the indicated days in culture the cells were fixed using four two-fold dilutions of 4% paraformaldehyde with 1 mM MgSO₄. Cells were allowed to fix in the final dilution for 20 minutes. The cells were then washed in PBSG for 10 minutes and then permeabilized and stored in 80% methanol over-night at −20° C.

In Situ Hybridization Probe Preparation.

In situ hybridization probe mixes were applied in aliquots of 50 μl per coverslip and assembled for each experiment from concentrated stocks. Once probe mixes were assembled, they were heated to 65° C. for a minute immediately prior to use. Primary nucleic acid element mix contained 2×SSC (300 mM NaCl, 30 mM Sodium Citrate), 10% dextran sulfate, 40% formamide, 0.1 μM of each primary nucleic acid for each gene hybridized, 20 μg/ml sheared salmon sperm DNA, 20 μg/ml E. coli RNAse free tRNA, 0.4% SDS. Secondary nucleic acid element mix contained 2×SSC, 10% dextran sulfate, 35% formamide, 0.1 μM each secondary nucleic acid element corresponding to the primary nucleic acid element used, 20 μg/ml sheared salmon sperm DNA, 20 μg/ml E. coli RNAse free tRNA, 0.4% SDS. Tertiary nucleic acid element mix contained 2×SSC, 10% dextran sulfate, 20% formamide, 0.1 μM each tertiary nucleic acid used.

Assembly of a Humidified Chamber.

A piece of parafilm was spread in the bottom of a 15 cm plastic culture dish. 50 μl of in situ hybridization nucleic acid element solution was placed on the parafilm without leaving air bubbles, leaving enough distance between probes so that coverslips will not contact each other during incubation. Coverslips were placed onto drops of solution. Approximately 200 μl of PBS was added in the corner of each coverslip to keep humidity in the chamber during incubation. The lid on the culture was sealed on the vessel by wrapping it with parafilm around all the edges.

In Situ Hybridization.

Coverslips with fixed cells were incubated in 80% methanol from above were warmed to room temperature, and serially rehydrated by 5 successive 2-fold dilutions with 2×SSC/40% formamide, followed by one complete change into 2×SSC/40% formamide. After 5-minutes coverslips were placed into primary nucleic acid element mix (prepared as above) cells-side down in a humidified chamber as above and incubated 37° C. overnight. All steps from here forward were performed in a 37° C. warm room, and all reagents kept at 37° C. The coverslips were gently pried off the parafilm, individually placed cells-side up into separate wells of a 6-well culture dish with 3 ml of 2×SSC/40% formamide and then rocked gently for 15 minutes. We used a 2-D rocking platform for all washing set at 45 oscillations per minute. This wash was repeated for three 15-minute intervals, then buffer changed to 2×SSC, 35% formamide to equilibrate the coverslips for the secondary hybridization. Coverslips were placed cells-side down on a drop of secondary nucleic acid element mix (prepared as above) in a hybridization chamber. The chamber was sealed with parafilm and incubated 3 hours. The coverslips were gently pried off the parafilm, individually placed cells-side up into separate wells of a 6-well culture dish with 3 ml of 2×SSC/35% formamide and then rocked gently for 15 minutes. This wash was repeated for three 15 minute intervals and then the buffer changed to 2×SSC, 20% formamide to equilibrate the cover-slips for the tertiary hybridization. Coverslips were placed cells-side down on a drop of tertiary nucleic acid element mix (prepared as above) in a hybridization chamber. The chamber was sealed with parafilm, covered with aluminum foil and incubated 3 hours. The coverslips were gently pried off the parafilm, individually placed cells-side up into separate wells of a 6-well culture dish with 3 ml of 2×SSC/20% formamide and then rocked for 15 minutes. This wash was repeated for three 15 minute intervals then the buffer changed to 1×SSC, 0.05% Tween 20 and 300 nM DAPI and rocked for 15 minutes. Cells were rinsed two times in 1×SSC, then mounted in hard set anti-fade microscopy mounting medium according to manufacturer's recommendations, and used for microscopy.

Image Acquisition and Analysis of mRNA Dispersion and Polarization.

Epifluorescence micrographs were obtained using an epifluorescence microscope (Nikon TiE) using a Cool Snap HQ2 or QuantEM digital camera. To be able to compare images shown, fluorescence micrographs from the same wavelengths (Cy3 or Cy5) within an individual experiment were acquired with the same exposure time, and the display scales of the representative images from each condition were equalized. For mRNA distribution analysis, serial Z-sections (0.5 μm steps, between 5-7 μm total distances) were acquired and a maximum projection image was generated using the Nikon Elements software. For quantification of the polarization and distribution of mRNAs, a manual mask was generated using ImageJ and the dispersion and polarization indexes were calculated using the script described. Cells that contained bright STIC probe aggregates were not imaged for mRNA distribution analysis.

### Example 2

### In Situ Hybridization Probes Detect mRNA

In situ hybridization probes design starts with one primary nucleic acid element directed against 50 nucleotides of an mRNA of interest and a secondary nucleic acid element that hybridizes to a trimerized unique 35 nucleic acid recognition element tethered to the end of the 50 nucleic acid antisense portion of the primary nucleic acid element (FIG. 1A, ODN 1a+1b, and ODN 2a+2b). A tertiary nucleic acid element hybridizes to a secondary nucleic acid element through a different pentamerized unique 25 detection element at the end of the secondary nucleic acid element (FIG. 1A, ODN 3). The tertiary nucleic acid element is directly coupled to a detection probe (i.e., fluorescent dye) allowing detection of the RNA that the probe is bound to through the In situ hybridization probe (FIG. 1A). The three successive hybridizations use nucleic acid molecules of decreasing length, and thus of oligonucleotide complementarity to facilitate decreasing stringency for each step. Thus, preventing subsequent dissociation of previously hybridized nucleic acid elements.

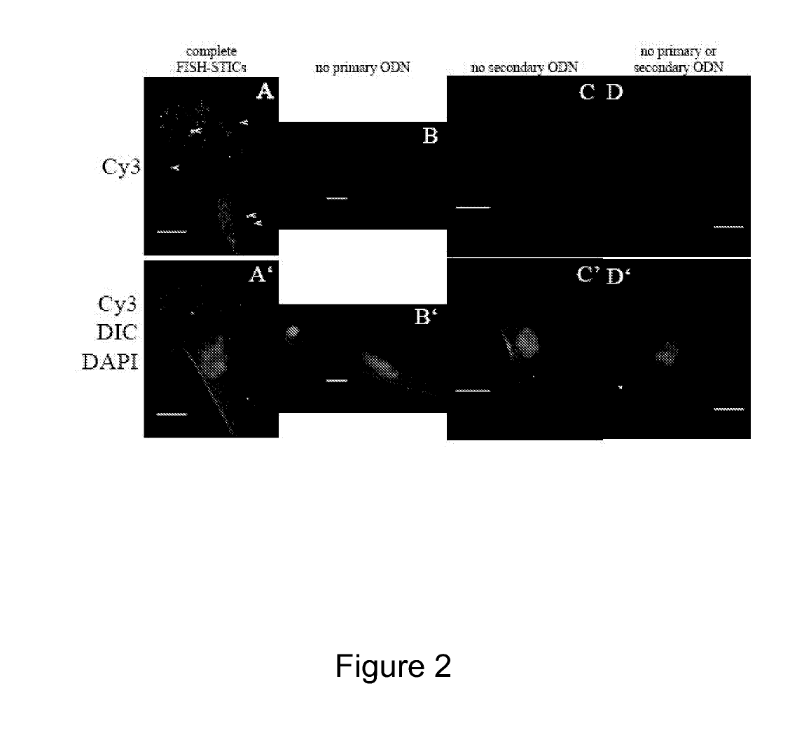

The β-actin (Actb) mRNA is a paradigm for an actively localized mRNA in cells and its distribution in cultured cells and primary neurons is governed by inadequately defined machinery that regulates the β-actin protein synthesis spatially and temporally. See Huttelmaier, S et al., Nature, (2005) 438(7067): 512-515; and Rodriguez, A. J. et al., J Cell Biol., (2006). 175(1): 67-76. The γ-actin (Actg) mRNA produces an almost identical protein and its distribution in cultured neurons is different from the Actb mRNA. See Bassell, G. J., J. of Neurosci. (1998) 18(1): 251-65. This shows that the Actg mRNA is regulated by different machinery than Actb mRNA. FISH-STICs probes were designed to identify single 50 nucleotide sequences within mouse Actb mRNA and hybridized these to immortalized MEFs on coverslips. These in situ hybridization probes labeled small fluorescent puncta in a primary, and secondary nucleic acid element dependent manner, demonstrating that fluorescence detection requires assembly of complete complexes, and that individual in situ hybridization probes are sufficient to detect mRNAs in cells (FIG. 2, compare panel A to panels B-D). These data show that in situ hybridization probes of the present disclosure form mRNA independent probe complexes that appeared brighter than individual mRNAs (FIG. 2A, arrowheads). However, the independent probe complexes did not form over cells or bare coverslip and Cy3 and Cy5 complexes did not co-localize in subsequent two-color hybridizations (FIG. 2). Thus, the appearance of these complexes was limited and their presence and intensity diminished with washing.

### Example 3

### The Use of a Second Primary Actb Probe that Binds the Same Secondary Nucleic Acid Element Intensifies the Fluorescent Signal

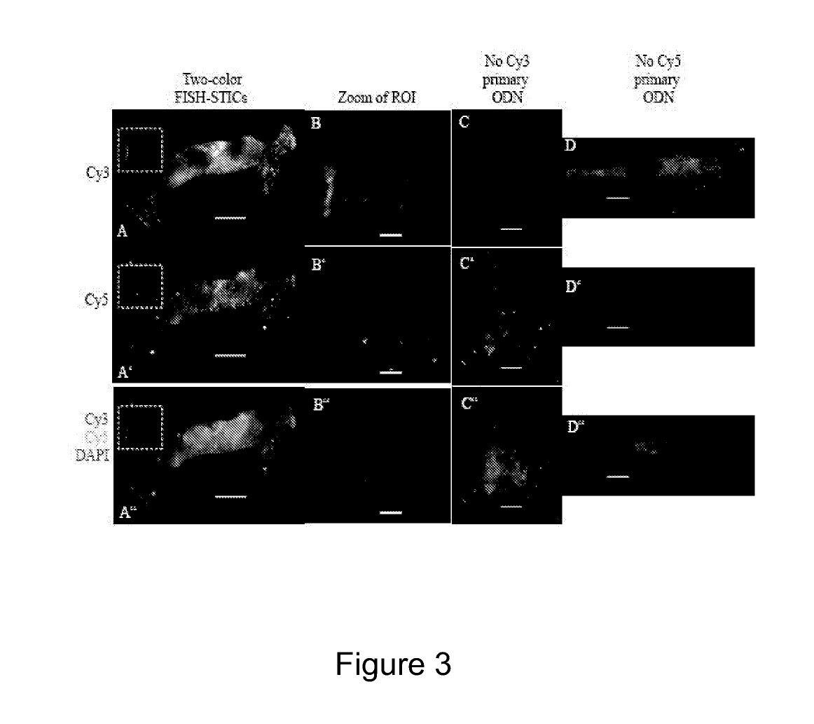

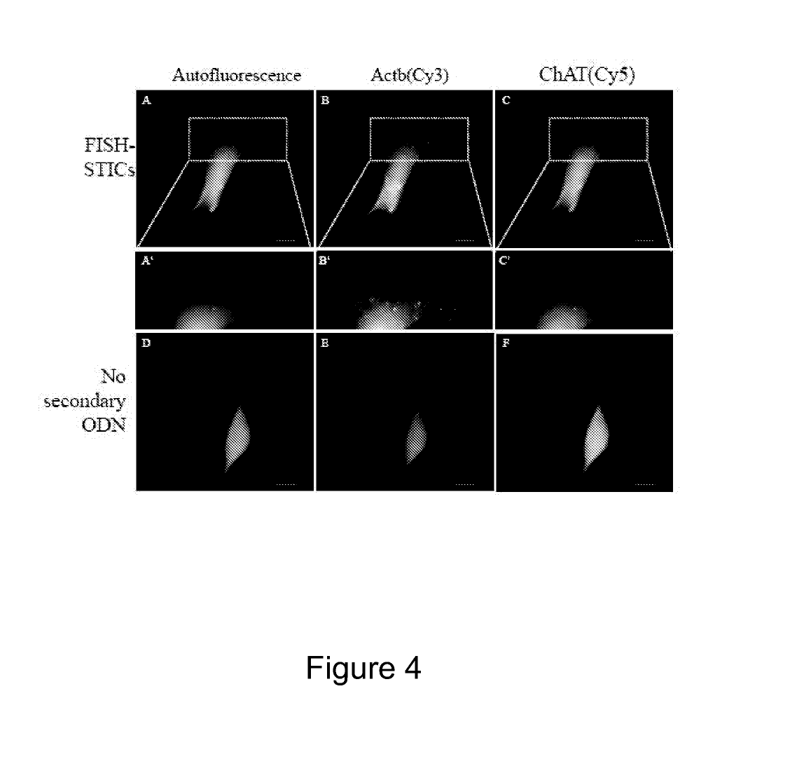

Single in situ hybridization probes could detect mRNA, but a stronger fluorescence signal is preferable for quantitative image analysis, so a second primary Actb nucleic acid element was created that would bind to the same secondary Cy3 nucleic acid element used previously. Two primary nucleic acid elements were designed for Actg mRNA that binds to a distinct secondary nucleic acid element and a Cy5 containing tertiary nucleic acid element for this secondary nucleic acid. Actb probes and Actg probes were co-hybridized to MEFs and Cy3 and Cy5 mRNA puncta were spatially separable, consistent with these in situ hybridization probes hybridizing to distinct mRNAs (FIG. 3, compare red and green overlap in panel A″ and panel B″). Different numbers of Cy3 and Cy5 puncta also apparent in the images, consistent with more Actb mRNA in these cells than Actg mRNA. To control for the specificity of hybridization in situ three primary FISH-STIC nucleic acid elements designed against the mouse choline acetyl-transferase (ChAT) mRNA were designed that would bind to the Cy5 secondary/tertiary nucleic acid element set. ChAT is not expressed in MEFs, and no specific hybridization could be detected, while the Actb mRNA labeled with Cy3 in the same MEFs appeared normal (FIG. 4). This control experiment demonstrates that the FISH-STICs probes of the instant disclosure are mRNA specific and do not have intrinsic background binding.

### Example 4

### FISH-STICs are Useful for Multi-Color mRNA Detection

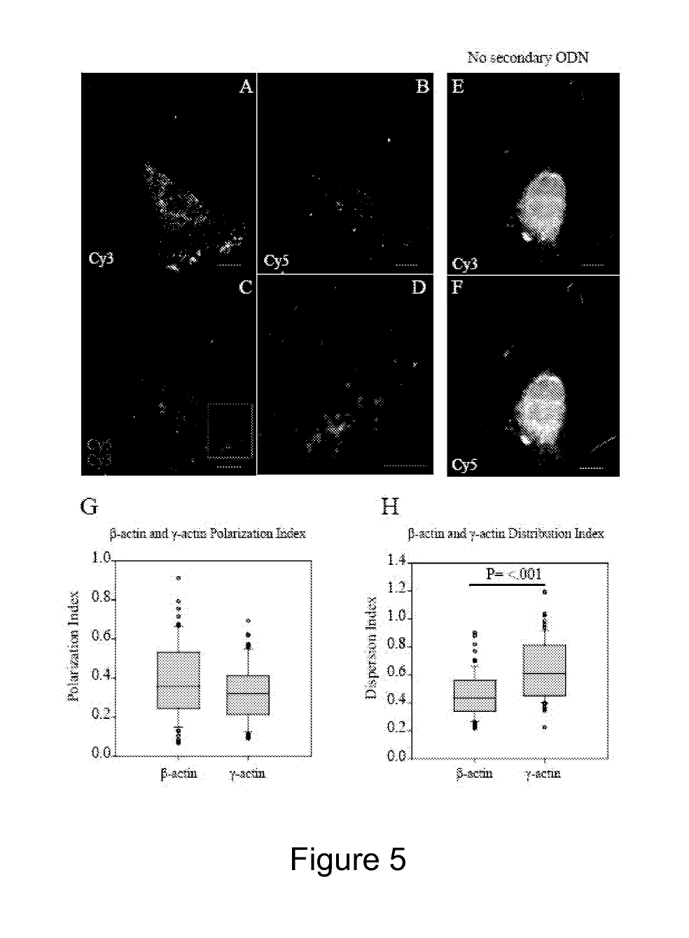

Next, FISH-STIC images were analyzed to quantify Actb and Actg mRNA distribution within the same cell by measuring the polarization index and dispersion index. The methods utilized in calculating the polarization index and dispersion index is previously described in Park et al., Cell Rep., (2012) 1(2): 179-84. These data show that Actb mRNA is more polarized within MEFs than Actg mRNA as reflected by its higher median polarization index (PI), (Actb=0.359, Actg=0.32, FIG. 5). However, Actg mRNA is more evenly distributed throughout the cell than Actb mRNA is reflected by its higher median dispersion index (DI) (Actb=0.434, Actg=0.609, FIG. 5). These results demonstrate the use of FISH-STICs for multi-color mRNA detection, and clearly establish that in mouse fibroblasts β-actin and γ-actin mRNA are under distinct regulatory processes that result in a quantifiably different distribution even in immortalized MEF cells.

### Example 5

### FISH-STIC Probes can be Used in any Cell Type

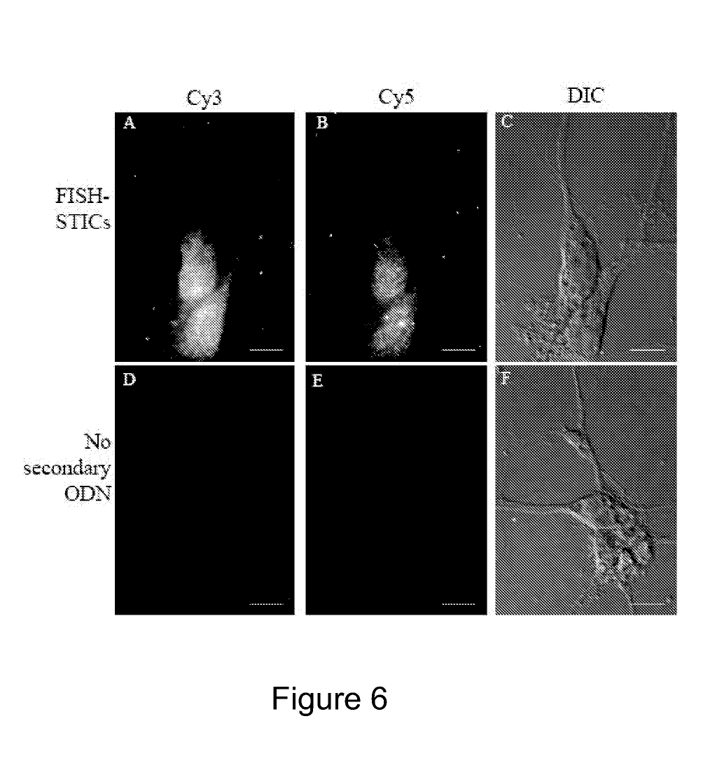

To investigate whether FISH-STICs could be used in other cell types three primary nucleic acid elements were designed against the mouse Type-III Neuregulin 1 (Nrg1-III) isoform mRNA that would bind to the Cy3 secondary/tertiary nucleic acid element set. The Nrg1 gene produces numerous isoforms within different tissues due to alternative promoters and splice sites and the type III isoform is neuron specific in the central nervous system. See Mei, L. and Xiong, W. C., Nat. Rev. Neurosci., (2008) 9(6): 437-52. Nrg1 nucleic acid molecules were hybridized to hippocampal neurons plated on poly-lysine coated glass coverslips. Nrg1-III and Actg mRNA were clearly detected in a secondary nucleic acid element dependent manner therefore the in situ hybridization probes and methods disclosed herein are applicable to any cell type (FIG. 6). The result also shows that the secondary and tertiary nucleic acid molecules used can also be successfully applied to primary nucleic acids that detect other mRNAs.

### Example 6

### Method of Using FISH-STIC Probes to Detect Nucleic Acids

Coverslips were prepared by placing acid washed sterile coverslips individually into wells of a 12-well tissue culture dish and rinse well with sterile H₂O. 18 mm coverslips fit comfortably into a well of a standard 12-well cell culture dish and leave enough room for handling. Cells grown on 18 mm glass coverslips that have been coated with standard cell culture grades of poly-L-lysine or extracellular matrix proteins are preferred, however any cell culture growth substrate has been shown to work without interfering with probe hybridization.

The prior H₂O wash is removed and coverslips are coated by adding 0.5 ml of 50 μg/ml PLK in BAB for at least 1 hour at room temperature. In certain embodiments coverslips can be prepared with this method the night before to allow the coating to develop.

The coverslips are then washed by changing solution in the wells to water and agitating to ensure the washes get to the underside of the coverslip. Coverslips are washed in this way at least 3 times with the third wash being removed leaving the coverslips without water prior to adding cells.

Initiate cell culture by detaching Mouse Embryonic Fibrobalsts (MEFs) from pre-culture and counting the cells. Notably, while MEFs have a suitably broad and flat morphology that allows for epifluorescence to generate good quality images, any cell type is amenable to use with the current methods, but cell morphology has an impact on the image acquisition. Broad and flat cells have less out of focus autofluorescence to provide less background fluorescence than bulbous cells that protrude high off the coverslip.

Seed

2.5×10⁴ to 5.0×10⁴ cells in 0.75 ml of D10 into each well of the 12-well dish and allow growth overnight. Cell density in the culture has an effect on cell morphology, with the cells in dense cultures being thicker than broad flat cells of sub-confluent cultures. Optimal results are obtained when most cells are sub-confluent and processed 24 hours after plating. Experimental cell culture manipulations or treatments can be performed on cells prior to fixation without interfering with RNA-FISH. If transfected cells are to be analyzed, transfection should be performed initially in a separate culture and then plated 24 hours post-transfection.

Cells are then fixed by fixing coverslips within the culture dish using four serial dilutions of PFA. Then the medium is removed so that only 0.5 ml D10 is left. Then 0.5 ml PFA is added with gentle mixing. This process was repeated until 4 two-fold dilutions have been performed, then all liquid was removed and PFA was added. While cell fixation was performed in 4% paraformaldehyde (PFA) in phosphate buffered saline (PBS) for 20 minutes, other fixatives that don't affect RNA can also be utilized in the current methods. In all cases, an effort to determine a fixation condition that results in as low an autofluorescence as possible for other cell types will yield the best results. After 20 minutes, remove PFA and replace with PBSG. After 10 minutes cells are permeabilized and stored in 80% methanol and kept at −20° C. overnight. Here cells are permeabilized after fixation in an alcohol solution (80% methanol), which also provides a secondary fixation. If cells are to be stored for an extended period of time the sample can be stored in alcohol solution at −20° C.

In another embodiment a first hybridization can be conducted in the same day as fixation by adding non-ionic detergent to the PBSG at a concentration well above the critical micelle concentration (0.1% Triton X-100 or IGEPAL-60) followed by several changes of 2×SSC without detergent. Cells can then be stored for 3-4 days after detergent permeabilizing.

Probe Preparation in accordance with the specification set forth in Table 2 shows the recipe for 50 μl Primary, Secondary and tertiary nucleic element solutions. One primary nucleic acid element solution droplet is provided per coverslip and generated in accordance with Table 2. Control hybridizations dropping out primary or secondary nucleic acid elements should be performed in parallel and water can be substituted for probe in those cases. The solution mixtures provided in Table 2 are designed to produce 50 W aliquots, which is ideal for 18 mm coverslips. Scaling up or down for other coverslip formats based on surface area is possible and does affect RNA-FISH

Rehydrate cells within the wells of the 12-well dish using 5 serial two-fold dilutions of 2×SSC with 40% formamide. After final dilution remove solution completely and replace with 2×SSC. Then a piece of parafilm was spread in the bottom of a plastic 150 mm culture dish to prepare a simple humidified chamber. The solution was then heated to 65° C. for 1 minute then one drop per coverslip was placed on the parafilm leaving suitable distance between drops so that coverslips do not contact each other during incubation.

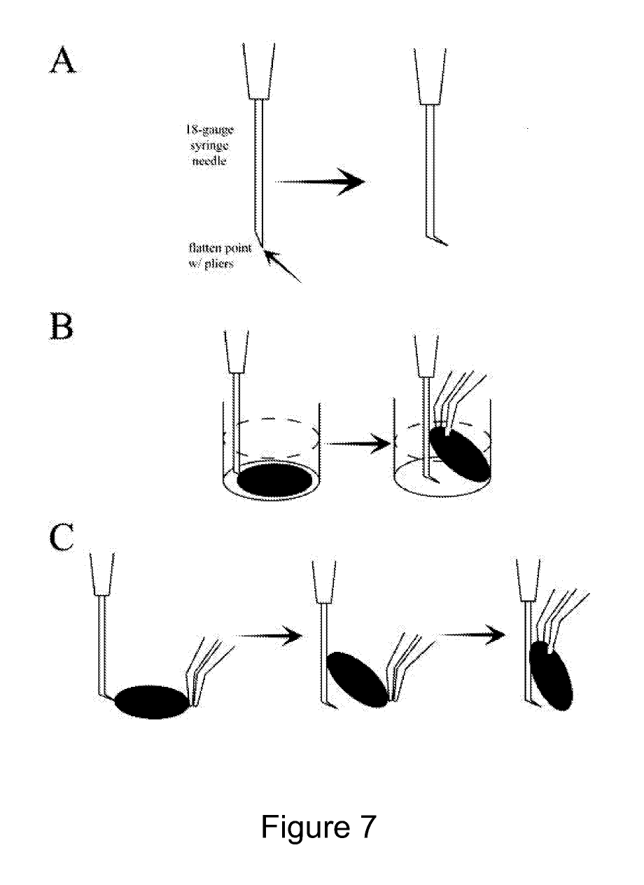

Coverslips with rehydrated cells were then removed from the wells and carefully placed cells-side down onto drops of solution, avoiding air bubble formations. Any method of carefully removing a coverslip can be used with the current methods, however the instant embodiment uses the pointed tip of an 18-gauge syringe needle, which was bent to a 90° angle and then grasping the bent end with pliers to bend it to 90° (FIG. 7A). These dislodging tools effectively help dislodge coverslips from the bottom of multi-well dishes (FIG. 7B) or from parafilm in the humidification chambers (FIG. 7C).

PBS was then added to the corner of each coverslip to keep humidity in the chamber during incubation and the culture dish was sealed by wrapping with parafilm. The cells were then incubated at 37° C. overnight. However, hybridization occurs so the primary solution incubation can be shortened to three hours, if desirable.

Next, the wash buffers are made and equilibrated overnight at 37° C.

The secondary and tertiary nucleic acid element solutions are then created in accordance with Table 2 and stored on ice until used. All steps from here forward are best performed at 37° C.

Six well dishes are prepared with 3 ml of primary wash buffer per well. Then coverslips are gently removed from the parafilm chambers and placed cells-side up into one well of a 6 well dish that has at least 3 ml of primary wash buffer. Coverslips are moved to 6-well dishes rather than 12-well dishes for washing to provide a larger volume and more agitation during washes because insufficient washing may result in the formation of large mRNA independent probe complexes that are suppressed by increased agitation.

Coverslips are then washed for 15 minutes by placing each dish on a 2D rocking platform set to 45 oscillations per minute. The wash buffer is changed completely for each of a second and a third wash. After the third wash, the buffer is changed to secondary wash buffer and incubated for 5 minutes. Then a piece of parafilm is spread in the bottom of a 150 mm plastic culture dish to prepare a simple humidified chamber.

The secondary nucleic acid solution is then heated to 65° C. for 1 minute and a 50 μl aliquot of secondary nucleic acid solution per coverslip is placed on the parafilm leaving enough distance between each droplet so that coverslips do not contact each other during incubation.

Remove coverslips from the wash vessel and carefully place each coverslip cells-side down onto a drop of secondary nucleic acid solution, avoiding air bubbles. Then add a PBS to the corner of each chamber to maintain humidity in the chamber during incubation, and seal the chamber by wrapping with parafilm and incubate at 37° C. three hours.

During incubation prepare a 6-well dish with 3 ml of secondary wash buffer per well and gently pry coverslips up from the parafilm and place them cells-side up into the wash vessel, one coverslip per well.

Coverslips are then washed for 15 minutes by placing each dish on a 2D rocking platform set to 45 oscillations per minute. The wash buffer is changed completely for each of a second and a third wash. After the third wash, the buffer is changed to secondary wash buffer and incubated for 5 minutes. Then a piece of parafilm is spread in the bottom of a 150 mm plastic culture dish to prepare a simple humidified chamber.

The tertiary nucleic acid solution is then heated to 65° C. for 1 minute and a 50 μl aliquot of tertiary nucleic acid solution per coverslip is placed on the parafilm leaving enough distance between each droplet so that coverslips do not contact each other during incubation.

Coverslips were then removed from the wash vessel and carefully place each coverslip cells-side down onto a drop of secondary nucleic acid solution, avoiding air bubbles. Then PBS was added to the corner of each chamber to maintain humidity in the chamber during incubation, and seal the chamber by wrapping with parafilm and incubate at 37° C. three hours.

Six well dishes are prepared with 3 ml of tertiary wash buffer per well. Then coverslips are gently removed from the parafilm chambers and placed cells-side up into one well of a 6 well dish that has at least 3 ml of tertiary wash buffer. Coverslips are moved to 6-well dishes rather than 12-well dishes for washing to provide a larger volume and more agitation during washes because insufficient washing may result in the formation of large mRNA independent probe complexes that are suppressed by increased agitation.

Coverslips are then washed for 15 minutes by placing each dish on a 2D rocking platform set to 45 oscillations per minute. The wash buffer is changed completely for each of a second and a third wash. After the third wash add DAPI stain buffer and incubate with agitation for 15 minutes. Next, the DAPI buffer is replaced by 1×SSC, which is subsequently replaced with fresh 1×SSC without incubation.

The coverslips are then mounted face down onto clean microscope slides using, for example, a hard-set anti-fade mounting medium, and allowed to cure. The mounted coverslips are then ready for imaging and data analysis. The instant methods view the samples under 60× and 100× magnification high numerical aperture objective lenses combined with standard scientific grade cameras that have pixel size between 6 and 8 μm provide sufficient magnification for image analysis with 100× providing slightly higher spatial oversampling. To acquire images for analysis, the exposure time and excitation intensities are set such that the images for the FISH and the negative controls are comparable. The exposure time and intensity required to obtain significant signal from the hybridized samples can be determined first, and the control samples containing no-probe can be acquired under the same excitation and exposure conditions.

TABLE 1
Oligonucleotides used

| | |
|---|---|
| Mouse Actb | 5′caacgaaggagctgcaaagaagctgtgctcgcgggtggacgcgactcTCGTTGGCCCCCG |
| primary1 | ACCGTTACAGACTGTTCTCAGTtcgttggcccccgaccgttacagactgttctcagtTC |
| SEQ ID NO: 1 | GTTGGCCCCCGACCGTTACAGACTGTTCTCAGT |
| Mouse Actb | 5′ggtggcttttgggagggtgagggacttcctgtaaccacttatttcatggaTCGTTGGCCCCCG |
| primary2 | ACCGTTACAGACTGTTCTCAGTtcgttggcccccgaccgttacagactgttctcagtTC |
| SEQ ID NO: 2 | GTTGGCCCCCGACCGTTACAGACTGTTCTCAGT |
| Mouse Actg | 5′ctccccagcccccaagtgaccgagccacatgaactaaggactaaatcaagTCTATAAACGA |
| primary1 | GCAATTACATAAGACATCCGTAGAtctataaacgagcaattacataagacatccgtag |
| SEQ ID NO: 3 | aTCTATAAACGAGCAATTACATAAGACATCCGTAGA |
| Mouse Actg | 5′tgacgagtgcggcgatttcttatccattgcgatcggcgaaggacTCTATAAACGAGCAAT |
| primary2 | TACATAAGACATCCGTAGAtctataaacgagcaattacataagacatccgtagaTCTA |
| SEQ ID NO: 4 | TAAACGAGCAATTACATAAGACATCCGTAGA |
| Secondary1 | 5′ACTGAGAACAGTCTGTAACGGTCGGGGGCCAACGAacgcgattgacta |
| (for Actb) | ccagactatacgACGCGATTGACTACCAGACTATACGacgcgattgactaccagac |
| SEQ ID NO: 5 | tatacgACGCGATTGACTACCAGACTATACGacgcgattgactaccagactatacg |
| Secondary2 | 5′TCTACGGATGTCTTATGTAATTGCTCGTTTATAGAtaccaattctgacata |
| (for Actg) | tgtgactcaTACCAATTCTGACATATGTGACTCAtaccaattctgacatatgtgactca |
| SEQ ID NO: 6 | TACCAATTCTGACATATGTGACTCAtaccaattctgacatatgtgactca |
| Tertiary1 | /Cy3/5′CGTATAGTCTGGTAGTCAATCGCGT |
| (for Actb) | |
| SEQ ID NO: 7 | |
| Tertiary2 | /Cy5/5′TGAGTCACATATGTCAGAATTGGTA |
| (for Actg) | |
| SEQ ID NO: 8 | |
| Nrg1-III | 5′tatgttccgctgccggaagcccatcgagagatgggtctgcactcagctgaTCGTTGGCCCCC |
| primary1 | GACCGTTACAGACTGTTCTCAGTtcgttggcccccgaccgttacagactgttctcagtT |
| SEQ ID NO: 9 | CGTTGGCCCCCGACCGTTACAGACTGTTCTCAGT |
| Nrg1-III | 5′agatcttctcggagttgaggcaccactgagacgctccgcttccaggcTCGTTGGCCCCCGA |
| primary2 | CCGTTACAGACTGTTCTCAGTtcgttggcccccgaccgttacagactgttctcagtTCG |
| SEQ ID NO: 10 | TTGGCCCCCGACCGTTACAGACTGTTCTCAGT |
| Nrg1-III | |
| primary3 | 5′cccccagggtcaaggtgggtaggagagtcgtattcgaatatcttgtccacTCGTTGGCCCCCG |
| SEQ ID NO: 11 | ACCGTTACAGACTGTTCTCAGTtcgttggcccccgaccgttacagactgttctcagtTC |
| | GTTGGCCCCCGACCGTTACAGACTGTTCTCAGT |
| Mouse ChAT | 5′ctcgctcccaccgcttctgcaaactccacagatgaggtactttgcagccTCTATAAACGAGC |
| primary1 | AATTACATAAGACATCCGTAGAtctataaacgagcaattacataagacatccgtagaT |
| SEQ ID NO: 12 | CTATAAACGAGCAATTACATAAGACATCCGTAGA |
| Mouse ChAT | 5′aacatgccagcttcatgtgagcccccaaggataggggagcagcaacaagcTCTATAAACGA |
| primary2 | GCAATTACATAAGACATCCGTAGAtctataaacgagcaattacataagacatccgtag |
| SEQ ID NO: 13 | aTCTATAAACGAGCAATTACATAAGACATCCGTAGA |
| Mouse ChAT | 5′gggggttataacaggaccatacccattgggtaccacagggccataacTCTATAAACGAGC |
| primary3 | AATTACATAAGACATCCGTAGAtctataaacgagcaattacataagacatccgtagaT |
| SEQ ID NO: 14 | CTATAAACGAGCAATTACATAAGACATCCGTAGA |

TABLE 2
Reagents and materials used in the current methods.

| | | | | Final |
|---|---|---|---|---|
| | Primary | Secondary | Tertiary | Concentration |
| 20xSSC | 5 μl | 5 μl | 5 ul | 2x |
| Salmon sperm DNA | 1 μl | 1 μl | 1 μl | 20 μg/ml |
| tRNA | 1 μl | 1 μl | 1 μl | 20 μg/ml |
| 10% SDS | 2 μl | 2 μl | 2 μl | 0.4% |
| Formamide | 10 μl | 7.5 μl | — | 40%, 35% or |
| | | | | 20% formamide |
| DS (in 40% formamide) | 25 ul | 25 μl | 25 μl | 10% |
| 5 μM nucleic acid | 1 μl | 1 μl | 1 μl | 0.1 μM |
| element | | | | |
| H₂O | 5 μl | 7.5 μl | 15 μl | — |

What is claimed is:

1. An in situ hybridization probe comprising:
a first nucleic acid element, wherein said first nucleic acid element comprises a nucleotide sequence portion that is complementary to a nucleic acid of interest, and a plurality of recognition elements that sequentially succeed the portion of the primary nucleic acid element having a nucleotide sequence that is complementary to a nucleic acid of interest;
a second nucleic acid element comprising an annealing element portion that is complimentary to the nucleotide sequence of said plurality of recognition elements present in said first nucleic acid element, and a plurality of detection elements that sequentially succeed the portion of said secondary nucleic acid element having a nucleotide sequence that is complementary to the nucleotide sequence of said plurality of recognition elements present in said first nucleic acid element; and
a third nucleic acid element comprising a nucleotide sequence complementary to the nucleic acid sequence of said detection elements present in said secondary nucleic acid element, and a detection probe.

2. The in situ hybridization probe of claim 1, wherein said nucleotide sequence complimentary to a nucleic acid of interest is about 50 nucleotides in length.

3. The in situ hybridization probe of claim 1, wherein said recognition elements are each about 35 nucleotides in length.

4. The in situ hybridization probe of claim 1, wherein said first nucleic acid element comprises 3 or more recognition elements.

5. The in situ hybridization probe of claim 1, wherein said first nucleic acid element comprises three recognition elements.

6. The in situ hybridization probe of claim 1, wherein said nucleotide sequence complementary to a nucleic acid of interest hybridizes to a nucleic acid of interest.

7. The in situ hybridization probe of claim 1, wherein said first, second and third nucleic acid elements are DNA.

8. The in situ hybridization probe of claim 1, wherein said first, second and third nucleic acid elements are RNA.

9. The in situ hybridization probe of claim 1, wherein said annealing element hybridizes to the recognition elements of the first nucleic acid element.

10. The in situ hybridization probe of claim 1, wherein said detection elements are each about 20 nucleotides in length.

11. The in situ hybridization probe of claim 1, wherein said second nucleic acid comprises 5 or more detection elements.

12. The in situ hybridization probe of claim 1, wherein said third nucleic acid element hybridizes to the detection elements of the second nucleic acid element.

13. The in situ hybridization probe of claim 1, wherein said detection probe is detectable by microscopy.

14. The in situ hybridization probe of claim 13, wherein said detection probe comprises cyanine dye.

15. The in situ hybridization probe of claim 15, wherein said cyanine dye is selected from the group consisting of Cy2, Cy3, Cy5 and Cy7.

16. The in situ hybridization probe of claim 1, wherein said detection probe is detectable by fluorescence activated flow cytometry.

17. A kit for detecting a nucleic acid of interest, the kit comprising a plurality of in situ hybridization probe as set forth in claim 1.

18. A method for manufacturing in situ hybridization probes for the detection of target nucleic acid sequences comprising:
selecting at least one nucleic acid of interest present in a sample;
synthesizing a primary nucleic acid element, wherein said primary nucleic acid element comprises a nucleotide sequence portion located at the 5′ end of the primary nucleic acid element that is complimentary to a portion of the at least one nucleic acid of interest and is capable of hybridizing to said at least one nucleic acid of interest, and said primary nucleic acid element further comprises at least one recognition element that sequentially succeeds the portion of the primary nucleic acid element having a nucleotide sequence that is complementary to a nucleic acid of interest;
synthesizing a secondary nucleic acid element, wherein said secondary nucleic acid element comprises a portion having an annealing element located at the 5′ end of the second nucleic acid that is complimentary to the nucleotide sequence of the of recognition elements present in said primary nucleic acid element, and a plurality of detection elements that sequentially succeeds the portion of the secondary nucleic acid element having an annealing element;
synthesizing a tertiary nucleic acid element, wherein said tertiary nucleic acid element comprises a nucleotide sequence that is complementary to the nucleic acid sequence of the detection elements present in said secondary nucleic acid element, and a detection probe; and
sequentially hybridizing the primary, secondary and tertiary nucleic acid elements to the nucleic acid of interest.

19. The method of claim 18, wherein said at least one recognition element comprises a first random nucleic acid sequence of 35 nucleotides.

20. The method of claim 19, wherein said first random nucleic acid sequence is created using a random sequence generator, and confirmed to lack complementarity with any endogenous nucleic acid sequence.

21. The method of claim 18, wherein said a plurality of detection elements comprises a second random nucleic acid sequence of 25 nucleotides.

22. The method of claim 21, wherein said second random nucleic acid sequence is created using a random sequence generator, and confirmed to lack complementarity with any endogenous nucleic acid sequence and said first random nucleic acid.

23. The method of claim 18, wherein said detection probe is detectable by fluorescent microscopy.

24. The method of claim 18, wherein said detection probe comprises a cyanine dye.

25. The method of claim 24, wherein said cyanine dye is selected from the group consisting of Cy2, Cy3, Cy5 and Cy7.
